(19) United States
(12) Patent Application Publication
(10) Pub. No.: US 2026/0069774 A1
(43) Pub. Date: Mar. 12, 2026

(54) DESIGN AND LOGIC TO DETECT INFUSION PUMP VERTICAL DISPLACEMENT AND MEASURE UNINTENDED BOLUS, MISSING VOLUME, TIME WITHOUT INFUSION, AND OTHER PARAMETERS

(71) Applicants: BAXTER INTERNATIONAL INC., Deerfield, IL (US); BAXTER HEALTHCARE SA, Glattpark (Opfikon) (CH)

(72) Inventor: Rajesh Swarnkar, Bangalore (IN)

(21) Appl. No.: 19/325,629

(22) Filed: Sep. 11, 2025

(30) Foreign Application Priority Data
Sep. 11, 2024 (IN) 202441068708

(51) Int. Cl. A61M 5/168

(52) U.S. Cl. CPC A61M 5/16854; A61M 5/16886; A61M 2005/16868; A61M 2205/18; A61M 2205/3306; A61M 2205/3379; A61M 2205/502; A61M 2240/00

(57) ABSTRACT

Improved medical pump designs, systems, and logic to detect medical pump vertical displacement are provided. The relative position of the pump with respect to an initial position set by a user may be measured. The designs, systems, and logic may also be used to detect the change in the height of a patient relative to the medical pump during therapy by measuring hydrostatic pressure in a patient line. The detected change in vertical displacements can be used to determine the volume of an unintended bolus or a missing volume during infusion therapy. The therapy may be adjusted based on the unintended bolus or missing volume as desired. The designs, systems, and logic may further include a transport mode to help maintain consistent therapy while a patient is ambulated or the pump is moved.

### PRIORITY CLAIM

This application claims the benefit of and priority to Indian Provisional Application No. 202441068708 titled “DESIGN AND LOGIC TO DETECT INFUSION PUMP VERTICAL DISPLACEMENT AND MEASURE UNINTENDED BOLUS, MISSING VOLUME, TIME WITHOUT INFUSION, AND OTHER PARAMETERS”, filed Sep. 11, 2024, the entire contents of which are hereby incorporated by reference.

### BACKGROUND

Medical infusion pumps can be used for the delivery of different medicaments in an in-patient or out-patient setting. Depending on the medicament being delivered and the type of pump used, bolus deliveries may be available for infusion. Because these medicaments are in liquid state, delivery is subject to principles of fluid dynamics. In the design of pumps it is typically presumed the pump will be kept at or near the level of a patient's infusion line insertion, or that the pump will generally be kept at a consistent height relative to the patient once and infusion is established, to maintain consistent fluid pressure and flow profiles. However, depending on patient needs and treatment necessities, pumps may need to undergo vertical displacement, inter alia, in order to ambulate the patient without stopping therapy or removing infusion lines. For example, a pump may need to be put onto a cart so that a patient can be wheeled to a different location. Similarly, patients may need to stand up or be lowered, which changes the level of the pump relative to the patient and vice versa.

In traditional infusion pumps, this vertical displacement causes unintended or excess bolus delivery or causes a reduction or interruption in fluid flow. In the former case, medicament is delivered in excess or too quickly to a patient. In the latter case, either the patient does not receive the entire medicament treatment or a catch-up dose must be delivered. This can lead to further problems, such as the treatment taking too long, and creates complications such as the need to determine the rate at which to deliver the catch-up dose, etc. At least one study has found that the period of interrupted flow is longer for neonatal patients (Syringe pump displacement alters line internal pressure and flow, Igarashi et al., 2005).

These systems also do not alarm or alert a user/clinician to interruptions in flow or unintended bolus caused by vertical displacement of the medical pump. These conditions may occur over the span of a few minutes.

Further still, medical pumps are not configured to implement a specific mode to deal with conditions caused by vertical displacement. A pump capable of adjusting to periods of no flow or periods of unintended bolus flow would markedly improve therapeutic efficacy.

Hence, improved designs and logic to be implemented on medical infusion pumps are needed.

### SUMMARY

The present disclosure relates to embodiments of a medical infusion pump designed to adapt to vertical displacement of the infusion pump or movement of the patient during therapy. The infusion pump may include non-transitory computer-readable logic configured to detect various conditions of the infusion pump and/or patient and to implement adaptive changes or cause alarms and/or alerts when displacement causes unintended consequences to patient therapy.

Given the need for accuracy, it may also be advantageous to provide a graphical user interface (GUI) designed to alert a patient or clinician as to the relative change in height of an infusion pump and/or patient, alert a patient or clinician to a time of no-flow and missing volume, alert a patient or clinician to a volume of unintended extra bolus, provide an input module or indicator of a transport mode being active, and/or provide an input module for a clinician to amend certain parameters of an ongoing or future therapy, among other existing GUI features.

In this regard, a first aspect described herein includes a system for measuring the vertical displacement of a medical pump, the system comprising a medical pump comprising a processor, a memory, and at least one sensor in operable communication with the processor, wherein the at least one sensor is configured to detect a change in height of the medical pump, wherein the memory stores data which correlates change in height of a medical pump to times of no flow or volumes of unintended boluses, and wherein the processor determines a time of no flow or a volume of an unintended bolus based on the detected change in height of the medical pump.

A further aspect includes wherein the at least one sensor includes a laser disposed on a bottom of the medical pump. The medical pump may use a laser triangulation method to determine the change in height of the medical pump.

In a further aspect, the at least one sensor further includes an accelerometer disposed within or on the medical pump and configured to detect movement of the medical pump. The medical pump may be configured to turn on the laser when movement of the medical pump is detected using the accelerometer. The accelerometer may also be configured to determine the vertical displacement of the medical pump based on acceleration data separate from a laser sensor.

A change in internal pressure of fluid in the patient line, such as the hydrostatic pressure of the fluid, during therapy may be used to further determine or confirm a time of no flow or a volume of an unintended bolus. The change in internal pressure can be measured by, for example, in the case of a syringe pump, a full bridge strain gauge force sensor disposed in a drive head which interfaces with the plunger of the syringe in the syringe pump. This sensor may also be used as a downstream occlusion sensor. Hydrostatic pressure in the patient line is translated to the syringe plunger upstream, which in turn exerts pressure on the drive head and on the pressure sensor disposed at the interface of the drive head and the syringe plunger. The medical pump may be configured to determine when a change in measured pressure is due to a downstream occlusion or due to a change in the relative height of the patient only.

In another aspect, the medical pump further comprises a graphical user interface. The graphical user interface is configured to display at least one of a change in relative height of the medical pump, a time of no flow, a missing volume, or a volume of unintended bolus. The graphical user interface may also provide further instrumentality, such as an alert to a change in relative height and/or a user input to allow an operator to adjust an ongoing therapy based on the change in relative height. The medical pump may cause an adjustment to an ongoing therapy to counteract effects of the change in relative height after a set period of time unless a user input is received on the graphical user interface.

In yet another aspect of the present disclosure, the medical pump is configured to include a transport mode. The transport mode may cause the medical pump to automatically adjust infusion therapy to counteract effects of the change in relative height at a fastest processing speed of the processor based on the stored data of the memory. The graphical user interface may be configured to display when the transport mode is active, and the transport mode may be configured to run for a set period of time.

Another aspect of the present disclosure includes wherein the at least one sensor is configured to detect a change in relative height of a patient connected to the medical pump during therapy. For example, a patient standing up or laying down creates excess pressure or causes a loss of pressure in the upstream direction of the line. The hydrostatic pressure may be translated to a sensor disposed at an upstream position of the pump. The medical pump may be configured to generate at least one of an alarm, an alert, a notification, or a pop-up when a change in the relative height of the patient is detected by the at least one sensor.

In another aspect, the data stored in the memory of the medical pump includes data specific to neonatal patients. The medical pump may be configurable to run in a neonatal patient mode, wherein the medical pump utilizes the data specific to neonatal patients while in the neonatal patient mode. The neonatal configuration may include settings more sensitive to patient movement or pump movement to avoid periods of no flow or unintended boluses, which may have negative clinical effects on a child patient.

Any of the features and aspects identified may be combined with any other feature or aspect. Numerous additional features and advantages of the present invention will become apparent to those skilled in the art upon consideration of the embodiment descriptions provided hereinbelow.

### BRIEF DESCRIPTION OF THE FIGURES

FIG. 1A illustrates an embodiment of a medical pump wherein the pump is experiencing vertical displacement according to the present disclosure.

FIG. 1B illustrates an embodiment of a medical pump wherein the medical pump is coupled to a patient experiencing vertical displacement according to the present disclosure.

FIG. 2A illustrates an embodiment of a medical pump coupled to an IV pole wherein the medical pump is coupled to a patient experiencing vertical displacement according to the present disclosure.

FIG. 2B illustrates an embodiment of a medical pump wherein the medical pump is coupled to a neonatal patient experiencing vertical displacement according to the present disclosure.

FIG. 3A illustrates a medical pump hydrostatic pressure over time during an infusion according to the present disclosure.

FIG. 3B illustrates another embodiment of a medical pump flow over time during an infusion according to the present disclosure.

FIG. 4 illustrates an embodiment of a medical pump wherein the medical pump utilizes laser vertical displacement detection according to the present disclosure.

FIG. 5A illustrates an embodiment of a medical pump wherein the medical pump includes a transport mode display on a graphical user interface according to the present disclosure.

FIG. 5B illustrates an embodiment of a medical pump wherein the medical pump is configured to display an alert on a graphical user interface according to the present disclosure.

FIG. 6A illustrates an example syringe and tubing setup of a medical pump according to the present disclosure showing a direction of deformation for certain components during an unintended bolus.

FIG. 6B illustrates the example syringe and tubing setup of FIG. 6A showing a direction of deformation for certain components during a period of reduced flow.

FIG. 7 illustrates a schematic representation of a system for determining vertical displacement of a medical pump and making therapy adjustments according to the same.

FIG. 8 illustrates a flowchart of an example method of determining vertical displacement of a medical pump and making therapy adjustments according to the same.

### DETAILED DESCRIPTION

While the invention is susceptible to various modifications and alternative forms, specific embodiments thereof have been shown by way of example in the drawings and are herein described in detail. It should be understood, however, that it is not intended to limit the invention to the particular form disclosed, but rather, the invention is to cover all modifications, equivalents, and alternatives falling within the scope of the invention as defined by the claims.

### Vertical Displacement Measurement-Pump

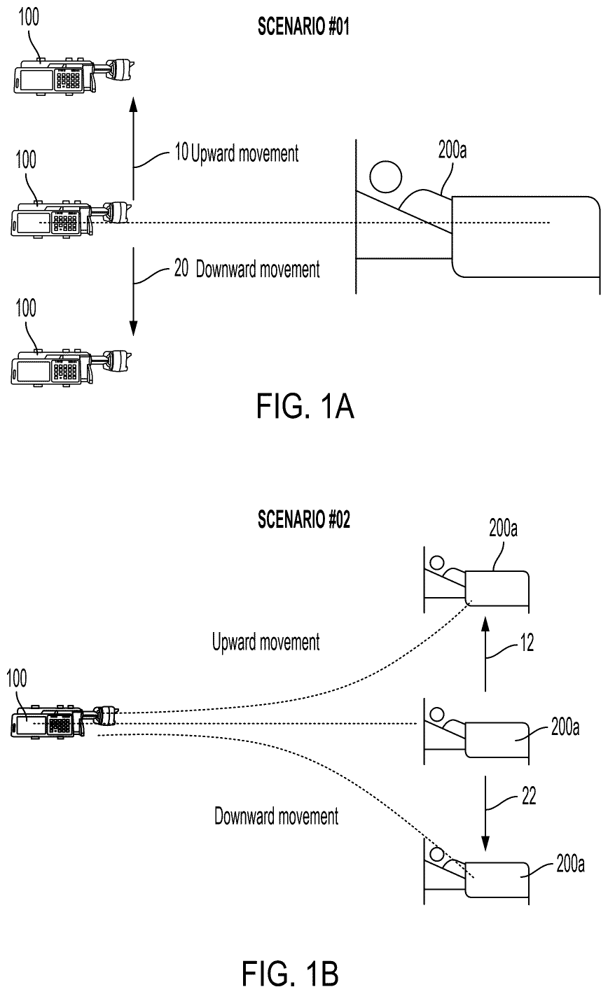

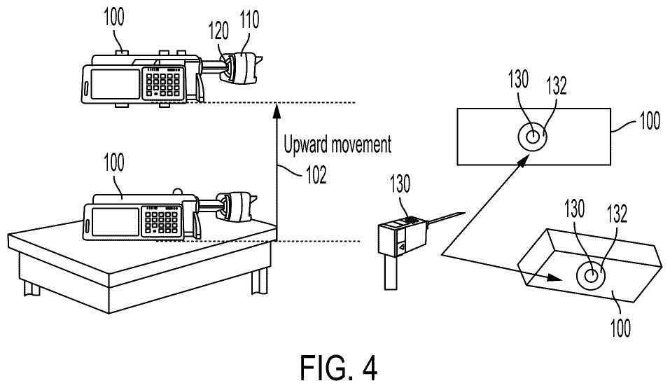

Turning to FIGS. 1A, 3A, 3B, and 4, a medical pump 100 is operably coupled to a patient 200a for therapy. The medical pump 100 in this embodiment is a syringe pump, but various other types of medical pumps (such as large volume pumps, PCA pumps, ambulatory pumps, etc.) are also contemplated. The medical pump 100 is ideally at or near the level of the patient's 200a infusion line insertion and remains so during therapy. In a preferred embodiment, the infusion line insertion is at the level of the patient's 200a heart. However, due to various expediencies, it may be necessary to move the medical pump 100 while infusion is occurring. This may require an upward movement 10 or a downward movement 20 of the medical pump. In a case where the medical pump 100 is subjected to downward movement 20, gravity works against the direction of flow of the fluid, causing an increase in hydrostatic pressure as the pump needs to work more against gravity. On the inverse side, when the medical pump 100 is subjected to an upward movement 10, gravity works to pull the fluid in the line toward the patient, causing a decrease in hydrostatic pressure (hydrostatic pressure may also be referred to as back pressure). The medical pump 100 may include a pressure sensor 120 in a drive head 110 of the medical pump 100, the pressure sensor 120 capable of measuring pressure from the plunger 153 of a syringe or other medicament container. The pressure sensor 120 is preferably a full bridge strain gauge force sensor, and the pressure sensor 120 may be used also as a downstream occlusion sensor, although other types and configurations of sensors are contemplated.

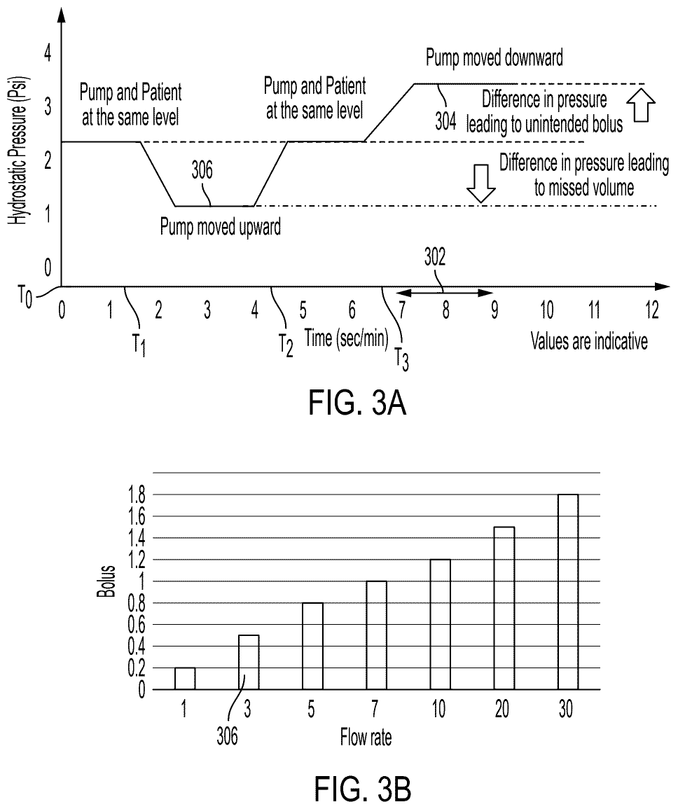

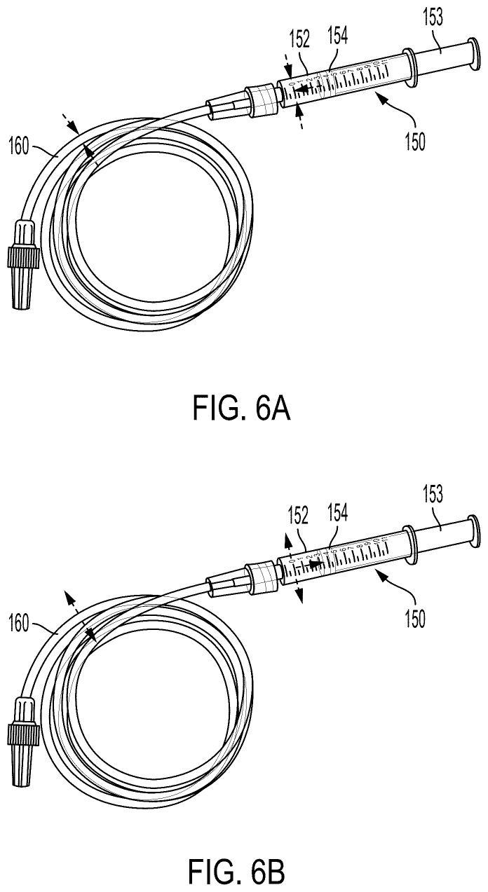

Medical pumps 100 are preferably configured to run with a constant flow rate. For instance, in the case of a syringe pump, the drive head 110 is typically configured to move the plunger 153 of the syringe at a constant rate. A negative change (decrease) to the hydrostatic pressure may indicate more medicament being infused than intended, because the components of the infusion line are typically made of flexible or semi-flexible materials. For example, a decrease in hydrostatic pressure (due to gravity pulling the fluid downward toward the infusion point of the patient 200a), may cause components of the medical pump 100, such as the tubing 160 and the syringe barrel 152 to contract and plunger rubber 154 of the syringe to expand into the barrel 152 of the syringe 150, decreasing the volume in the infusion line and causing a temporary bolus or excess flow, as illustrated in FIG. 6A. As shown in FIGS. 6A-6B, dashed arrows indicate an example direction of displacement for some flexible and semi-flexible components of the medical pump 100. The medical pump 100 may experience a decrease in the amount of pressure sensed by the pressure sensor 120 in such a scenario, since the drive head 110 will continue to move at a constant rate. By detecting the change in height of the medical pump 100 at T₁, an unintended bolus 304 can be inferred based on known data sets which correlate vertical displacement to changes in hydrostatic pressure. Once the pump is returned to the level of the patient infusion line insertion (or its original vertical position) or the pump has a chance to reach steady state again such as at time T₂, the infusion will continue at its original setting, but with an excess unintended bolus 306 volume having been delivered to the patient. FIG. 3B describes the volume of unintended bolus compared to the flow rate of the medical pump 100 infusion line. Although only one label is used in FIG. 3B, all bars in the graph represent a different unintended bolus amount 306 compared to the flow rate. This changes the infusion volume to a second actual infusion volume in the scenario of FIG. 3A, with a second constant volume difference to the intended infusion volume.

A positive change (increase) to the hydrostatic pressure, on the other hand, may indicate less medicament being infused than intended. The illustration of FIG. 3A models this missing volume 304 from an increase in hydrostatic pressure in the infusion line as shown beginning at T₃, where a time of no infusion 302 has occurred due to the excess pressure in the line, which may cause components of the medical pump 100, such as the tubing and the syringe barrel 152 to expand and plunger rubber 154 of the syringe to contract outward from the barrel 152 of the syringe, increasing the volume in the infusion line and causing a missing volume 304, as shown by the dashed arrows indicating displacement of the flexible and semi-flexible components of the medical pump 100 in FIG. 6B. The medical pump 100 may experience an increase in the amount of pressure sensed by the pressure sensor 120 in such a scenario, since the drive head 110 will continue to move at a constant rate despite the relatively excess pressure. Once the condition has stabilized or the medical pump 100 has returned to its original vertical position the infusion may continue at its original setting, but with a missing volume 304 in the therapy caused by the period of vertical displacement. In some instances, a patient will experience both a time of no flow 302 and an unintended bolus 306, such as in FIG. 3A.

The vertical displacement and rate of vertical displacement may be measured by a laser sensor 130 mounted preferably to the bottom of the medical pump 100 and protected by a protective glass 132. The laser sensor 130 may employ laser triangulation methods to measure the upward movement 10 of the medical pump 100. In a preferred embodiment, a user manually or the pump automatically configures a baseline measurement for the laser sensor 130 from which to measure vertical displacement. The baseline measurement may be based on the medical pump's 100 location from the floor or from a surface below the medical pump 100. In a preferred embodiment, the rate of vertical displacement, if desired, can be measured by an accelerometer 170 disposed inside of the medical pump 100 with the ability to measure acceleration along at least a vertical axis, but more preferably along three orthogonal axes. In an alternative embodiment, the rate of vertical displacement may be calculated using the vertical displacement data provided by the laser sensor 130. Comparing the upward movement 10 and/or rate of upward movement 10 to known or measured data sets yields a determination of either a time of no flow 302 or an unintended bolus 306 which can be used by a processor 190 of the medical pump 100 to determine the difference between the intended infusion volume at a given time and the actual infusion volume at that time. In an alternative embodiment, an accelerometer 170 configured to sense movement of the medical pump 100 may be used in order to start and stop the laser sensor 130, so as to conserve power by not having the laser sensor 130 constantly on. This accelerometer 170 may be the same accelerometer 170 that measures the rate of vertical displacement of the medical pump 100. In an alternative embodiment, the accelerometer 170 may measure the upward movement 10 on its own, without the need for a laser sensor 130 at all. The medical pump 100 may be configured to make flow rate adjustments based on these measured displacements and associated excess or lacking volumes, as described in further detail below.

### Vertical Displacement Measurement—Patient

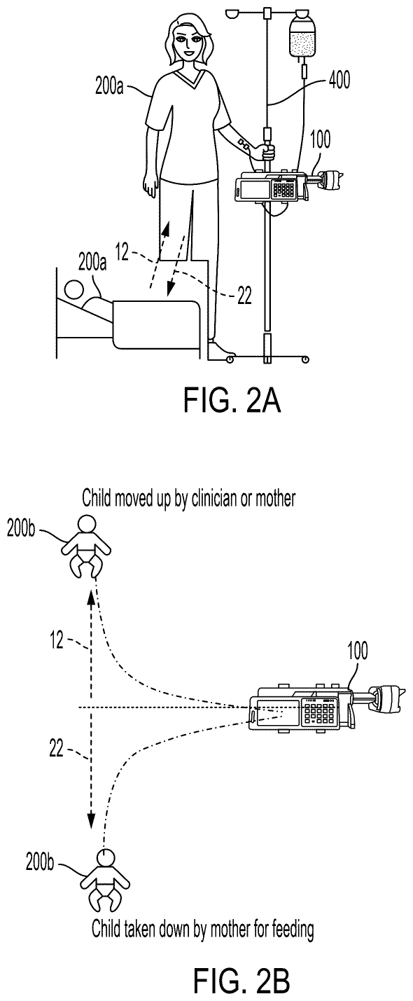

The above description measured the vertical displacement of the medical pump 100 relative to the patient 200a. Turning now to FIGS. 1B-2B, vertical displacement of the patient 200a relative to the medical pump 100 is described.

The patient 200a may be raised 12 or lowered 22 for various reasons while infusion therapy is carried out. This may be that the patient's 200a bed is raised for convenience, that the angle of the patient's 200a bed is increased in order to perform an assessment or collect vital information, the patient 200a is transferred to a raised platform or gurney to transport the patient 200a to a different room or for imaging to be performed, etc. The patient 200a may also ambulate to a different location, such as the lavatory, a different treatment room, for physical therapy reasons, etc. In such a scenario, the medical pump may be placed on or have already been affixed to an IV pole 400 or other moveable carrier/rack. The act of the patient 200a getting up or laying back down raises 12 or lowers 22 the level of the patient 200a (specifically, the point at which the patient line is placed in the patient 200a) relative to the position of the medical pump 100. Such displacement can cause hydrostatic pressure in the line to increase or decrease, depending on the direction of displacement.

Further, pediatric or neonatal patients 200b may be raised 12 or lowered 22 for various reasons while infusion therapy occurs, such as for feeding, changing diapers, because the movement of the neonatal patient 200b is not practicably controllable, for necessary play time, etc. It has been found that infusion therapy is more interrupted when used for a neonatal patient 200b than for an adult patient 200a, and that the effects of interrupted flow or unintended bolus flow can be more severe for the neonatal patient 200b. For example, an unintended bolus of up to 2% has been measured on existing medical pumps 100, which an adult patient 200a may be able to handle but may cause severe reactions in a neonatal patient 200b, due to differing blood and body fluid volumes (the same unintended bolus will likely result in a larger percentage change to the fluid composition and concentrations of a neonatal patient 200b than to an adult patient 200a).

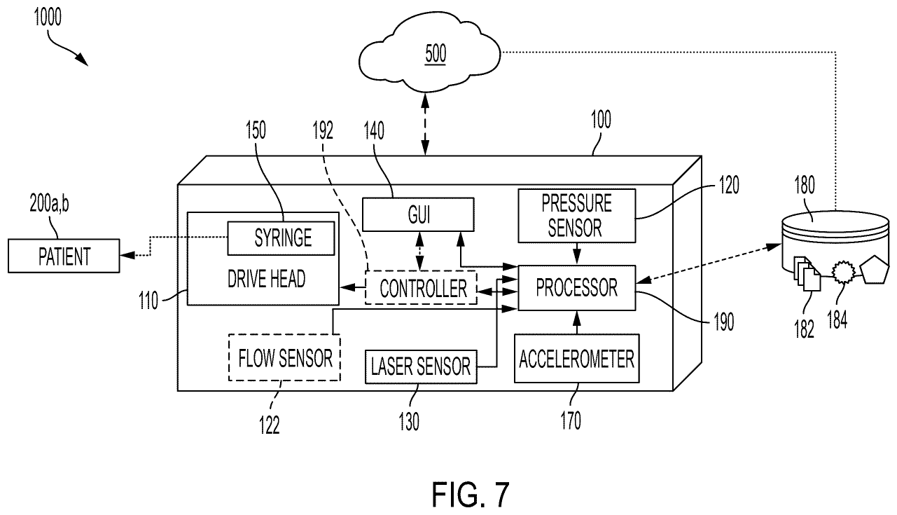

In general, similar times of no flow 302, missing volumes 304, and unintended boluses 306 occur due to raising 12 and lowering 22 of the patient 200a, 200b as shown in FIGS. 3A-3B as with upward movement 10 and downward movement 20 of the medical pump 100. The effect may be inverse as to raising 12 of the patient 200a, 200b and lowering 22 of the patient 200a, 200b as it is to upward movement 10 and downward movement 20 of the medical pump 100 because the relative effect of the movements are inverse (i.e. upward movement 10 of the medical pump 100 causes the same relative change in position to the patient 200a, 200b as lowering 22 the patient 200a, 200b does). However, various adjustments can be made depending on further data collected as to the correlation between certain height changes and different flow conditions, and differences between these when the medical pump 100 is moved or the patient 200a, 200b is moved. Further, it has been found that neonatal patients 200b experience longer times of no flow 302, so a separate neonatal patient 200b data set may be employed in certain circumstances. The medical pump 100 may be configured to allow a clinician or patient 200a, 200b to initiate a neonatal mode that uses the neonatal patient 200b data set rather than the adult patient 200a data set. The medical pump 100 may also be configured to update the data used for flow determinations either by connection to a network (for example network 500 in FIG. 7), by data imported from an external drive, or by manual data input. Data imported from an external drive and/or manually input may be stored in a memory 180 of the medical pump 100, and manual data input may be conducted by an input on the GUI 140, as schematically illustrated in FIG. 7.

Vertical displacement of the patient 200a, 200b may be measured by various sensors on the medical pump 100. As stated previously, the medical pump 100 may be a syringe pump which includes a pressure sensor 120 in the drive head 110, the pressure sensor 120 capable of measuring hydrostatic pressure in the patient line translated to the plunger 153 of a syringe. The pressure sensor 120 is preferably a full bridge strain gauge force sensor, and the pressure sensor 120 may be used also as a downstream occlusion sensor, although other types and configurations of sensors are contemplated. Unexpected or excess hydrostatic pressure in the upstream direction or a drop in hydrostatic pressure in the upstream direction caused by movement of the patient will be translated from the fluid to the syringe plunger 153, in which case the pressure sensor 120 will detect pressure from the syringe plunger 153. The sensor 120 is in operable communication with the processor 190 of the medical pump 100 and can relay data from pressure experienced from the syringe plunger 153. This data can be used to determine the time of no flow 302, the missing volume 304, and/or the unintended bolus 306 when no vertical displacement of the medical pump 100 is measured or less vertical displacement of the medical pump 100 is measured than would be expected with the change in hydrostatic pressure (such as if the medical pump 100 and the patient 200a, 200b both move at the same time). Typical pressure profiles for vertical displacement of a patient 200a, 200b tend to have a sudden absolute change in pressure including a period of high fluctuation, followed by a linear stabilization once the patient 200a, 200b reaches rest. The linear stabilization in pressure may be at a higher or lower level than the pressure when the medical pump 100 is at the level of the patient's 200a, 200b infusion line insertion. The sudden changes in pressure with a period of high fluctuation, shortly followed by a linear stabilization is one way in which the medical pump 100 can distinguish this condition from, for example, a downstream occlusion. In a downstream occlusion scenario, the pressure tends to continue to build or stay at a constantly high level, rather than have high fluctuation periods that resolve quickly. In this way, the medical pump 100 may be configured to distinguish between a downstream occlusion and a change in relative height of a patient 200a, 200b based on pressure sensor 120 data communicated to the processor 190.

In an alternative embodiment, these measurements may be done by included pressure sensors 120 or flow sensors 122 to measure pressure changes or flow changes in the patient line. The change in pressure or flow can be used to determine the change in patient 200a, 200b vertical displacement using known data as to the change in pressure or flow corresponding to vertical displacement. This may be useful in determining whether a patient 200a, 200b has moved or changed positions, which a clinician or hospital staff may want to correct.

### Clinician Adjustments

These changes in height and the corresponding time of no infusion 302, missing volume 304, and unintended bolus 306 may lead to unwanted hemodynamics, oxygenation, critical infusion delay, under-infusion, and/or over-infusion.

However, a clinician may determine in their best judgment that it would be better for a patient 200a, 200b to continue the infusion therapy on the current trajectory, rather than make adjustments to return the infusion therapy to the originally intended trajectory of volume infused and rate of infusion. This may be due to time constraints of the infusion therapy relative to other appointments the patient may have (such as imaging, respiratory therapy, etc.), or may be because the clinician believes enough medicament has been delivered such that a rectification of the infusion volume is not necessary and risks more harm than help. This may also be because more movement of the patient 200a, 200b or the medical pump 100 is expected before the end of therapy, and the patient 200a or clinician wishes to wait until all movement is done before causing an adjustment and/or correction to the therapy. Because of this, the medical pump 100 in the embodiments of FIGS. 1A-5B is configured to allow a clinician to choose whether an adjustment to therapy will be made or whether the therapy should continue as currently operating. The amount of infusion to add or subtract from the therapy in these adjustments, and the rate at which these adjustments are implemented may be based on data stored in a memory 180 of the medical pump 100 which correlates the vertical displacement data to a time of no flow or an amount of unintended bolus based on averaged data from multiple users.

In an alternative embodiment, the medical pump 100 may be configured to automatically adjust or correct the therapy. In another alternative embodiment, the medical pump 100 may be configured to delay an automatic adjustment or correction to the infusion therapy, giving the patient 200a or clinician a set amount of time to cancel or override the automatic adjustment/correction.

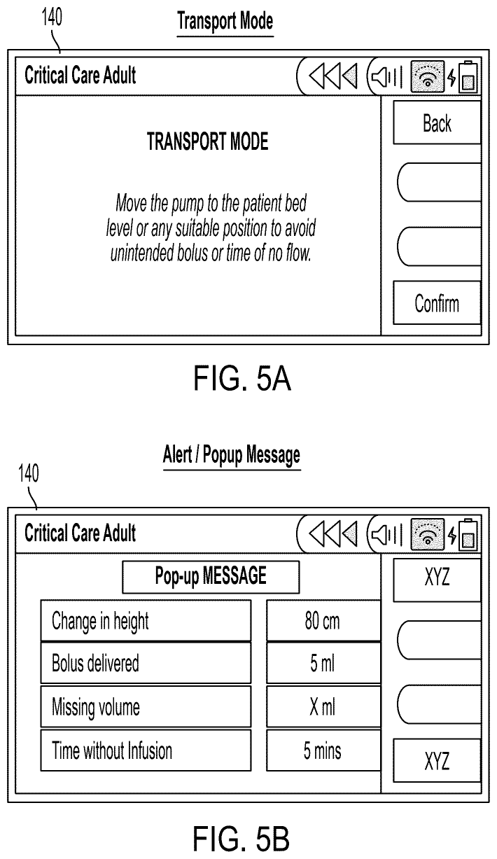

The medical pump 100 may allow the choices above by the patient 200a or clinician to be made by way of a graphical user interface (GUI) 140, which is described in more detail in the description of FIGS. 5A-5B, below, and may be implemented by a processor 190 of the medical pump 100 in communication with the laser sensor, pressure sensor 120, accelerometer 170, or other sensors and configured to control the flow of medication in the patient line.

### Transport Mode

The medical pump 100 may further include a transport mode, in which the medical pump 100 is configured to make real-time adjustments or corrections to the infusion therapy based on relative changes in height of the patient 110, 200b or the medical pump 100, or changes to line pressure or flow. This transport mode can mitigate or eliminate the instances of missing volume or unintended bolus by compensating for typical known changes to infusion volume as it happens. The transport mode can also direct the clinician or patient 200a, 200b to move the pump at a given time and to an intended position so that the medical pump 100 can increase a sampling time used to determine changes in patient 200a, 200b height or medical pump 100 height and thereby more accurately make adjustments or corrections in real time without always having this increased sampling rate (which would potentially cause increased energy usage or battery depletion).

As illustrated in FIG. 5A, the GUI 140 may include input means for a patient 200a, 200b to initiate the transport mode, and may display to a patient 200a, 200b or clinician that transport mode is active. The transport mode may run for a set period of time, or may run until canceled by the input of a patient 200a, 200b or clinician. The medical pump 100 may be configurable to allow for the patient 200a, 200b or clinician to choose whether the transport mode will run for a set period of time or whether the transport mode will run until canceled. The GUI 140 may also display the parameters and adjustments (described in more detail below in the description of FIG. 5B) being made in real time or nearly in real time while in the transport mode, in order to keep the patient 200a, 200b and/or clinician updated as to the operation of the medical pump 100. Further, in an alternative embodiment, the transport mode can be configured to run in only one direction, meaning that it will only cause adjustments to the infusion therapy if there is an unintended bolus or a missing volume as desired, but not both. The patient 200a, 200b or clinician may be able to decide in this embodiment which direction the transport mode will run in, and may be able to change this at a later time. For example, if a clinician decides that unintended boluses pose an insignificant risk but that missing volumes pose a significantly high risk, the clinician can configure the medical pump 100 to only make adjustments/corrections to infusion therapy when a missing volume occurs.

### Alerts, Alarms, and/or Notifications

As shown in FIG. 5B, the medical pump 100 may be configured to display alerts, alarms, pop-ups, and/or notifications on the GUI 140. The alerts may include displaying the relative change in height of the patient 200a, 200b and/or the medical pump 100. The alerts may also include displaying parameters such as change in relative heights, missing volumes, times of no flow, volume of bolus delivered, and/or other data relevant to a clinician or patient 200a, 200b in deciding whether to make any changes to the infusion therapy. This information may also be stored in a memory 180 of the medical pump 100 by the processor 190 or otherwise as part of a patient history or patient log.

The GUI 140 may also display input options to a clinician after a change in relative height occurs in order to make changes including adjustments or corrections to the infusion therapy. Further, as described above, the GUI 140 may generate an alert that an adjustment or correction to infusion therapy will automatically be made after a certain period of time unless canceled by input of a clinician or patient 200a, 200b. The GUI 140 may also provide input means for a clinician or patient 200a, 200b to override (change) or cancel an ongoing adjustment/correction if the clinician or patient 200a, 200b cannot reach the GUI 140 in time to cancel the operation before the time period expires or if the clinician or patient 200a, 200b changes her mind and no longer wants the adjustment/correction to be made.

### Example System

Turning now to FIG. 7, an example of a medical pump 100 system is illustrated schematically in order to highlight features of the medical pump 100 which allow for the above-described functions. It will be appreciated by those of skill in the art that not all components of the medical pump 100 system are necessary, and further optional components may be added as desired to allow for other functions or enhanced functionality of the medical pump 100. The illustrative features should not be construed as exhaustive except where claimed as such.

The medical pump 100 of the illustrated system 1000 for the detection of pump vertical displacement may be any appropriate medical pump 100 such as a large volume infusion pump, a syringe pump, an IV infusion pump, a peritoneal dialysis infusion pump, or another medical pump 100. The medical pump 100 illustrated as an example embodiment in FIG. 7 is a syringe pump including a drive head 110 which drives a syringe 150 to infuse a fluid in the syringe 150 into a patient 200a,b, as discussed in further detail above. The medical pump 100 further includes one or more sensors that are capable of measuring certain parameters of the medical pump 100. For example, a laser sensor 130 may be disposed in or on a housing of the medical pump 100. The laser sensor 130 of the system 1000 is in some embodiments disposed on a bottom part of the housing of the medical pump 100 such that the laser sensor 130 can be used to detect the vertical displacement of the medical pump 100. A processor 190 may be included in the medical pump 100 in operable communication with the laser sensor 130 such that data from the laser sensor 130 is sent to the processor 190, such that the processor 190 is able to make determinations on vertical displacement of the medical pump 100. Alternatively, the laser sensor 130 may have an integrated processor to make vertical displacement determinations instead of or in addition to the processor 190. A pressure sensor 120 may also be included as described previously in the medical pump 100. The pressure sensor 120 in some embodiments is included in or disposed on the drive head 110 and measures pressure between the drive head 110 and the syringe 150. The measurements (which may be in the form of data) from the pressure sensor 120 may be used by the processor 190 in operable communication with the pressure sensor 120 to make a determination of whether a downstream occlusion is occurring. The pressure sensor 120 may alternatively have an integrated processor to make downstream occlusion determinations instead of or in addition to the processor 190. A flow sensor 122 may optionally be included in the medical pump 100 and in operable communication with the processor 190. The flow sensor 122 may be used to measure flow through the tubing lines from the syringe 150 to the patient 200a,b and may thereby also aid in the determination of a downstream occlusion or in an instance of an unintended bolus or time of missing volume. An accelerometer 170 may be included as described previously in the medical pump 100 and in operable communication with the processor 190 in order to wake up the laser sensor 130 or to make vertical displacement determinations.

The processor 190 may be in operable communication with a controller 192 such that determinations based on data from the various sensors made by the processor 190 can be input to the controller 192 in order to cause a change in therapy or another output from the controller 192. For example, a determination of a downstream occlusion by the processor 190 based on data from the pressure sensor 120 may be sent to the controller 192, and the controller 192 in turn may cause an alert or popup message to be displayed on the GUI 140 of the medical pump 100. The GUI 140 may have input/output features (for example buttons) so that a user is able to respond to a prompt from the controller 192 for an action to be taken in response to a downstream occlusion, for instance. For example, the GUI 140 may be used for a user input for the medical pump 100 to enter the transport mode by sending a message to the controller 192 to start the transport mode. In some embodiments, the controller 192 and processor 190 may be integrated into a single unit to perform the functions of each component.

In some embodiments, a memory 180 is included internal to or externally to the medical pump 100 and in operable communication with the processor 190. The memory 180, as described above, may include various logs and/or tables 182 correlating a vertical displacement amount to a volume of unintended bolus or time of no flow in order for a catch-up adjustment to be made. The memory 180 may also store non-transitory machine-readable instructions and/or software 184 to be run by the processor 190 for the various medical pump 100 functions. The medical pump 100 and/or memory 180 may be in communication with a network 500. The network may allow for updates to the logs/tables 182 and the software 184 to be sent to the memory 180.

### Example Methods

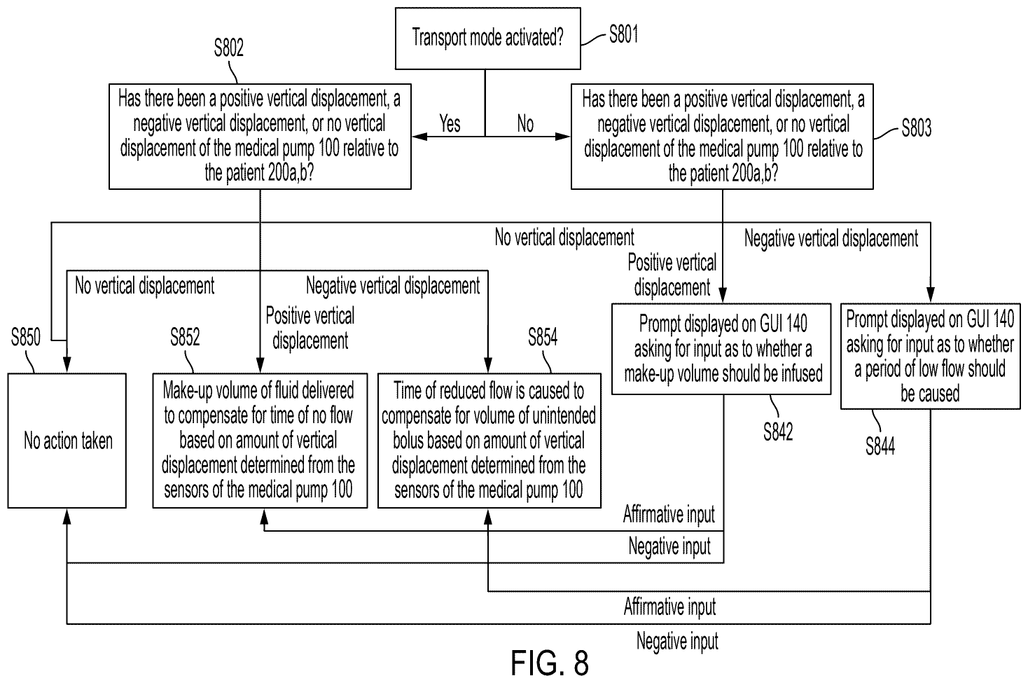

FIG. 8 illustrates an example method 800 for detection of a vertical displacement of a medical pump and an adjustment to therapy based on the same. The method starts at first step S801 where the medical pump 100 determines whether a transport mode is activated. The medical pump 100 may determine this, for example, by the processor 190. If the transport mode is activated, the method 800 proceeds to step S802 where it is determined whether a positive (upward) vertical displacement of the medical pump 100 relative to the patient 200a,b has occurred, whether a negative (downward) vertical displacement of the medical pump 100 relative to the patient 200a,b has occurred, or whether no vertical displacement has occurred. The determination may be made, again for example, by the processor 190. If it determined that no vertical displacement has occurred, the method 800 proceeds to step S850, where no action is taken. If it is determined that a positive vertical displacement has occurred, the method 800 proceeds to step S852 where a make-up volume of fluid is delivered to compensate for the time of no flow based on the amount of vertical displacement determined from the sensors of the medical pump 100. If it is determined that a negative vertical displacement has occurred, the method proceeds to step S854 where a time of reduced flow is caused to compensate for the volume of unintended bolus based on the amount of vertical displacement determined from the sensors of the medical pump 100.

If the transport mode is not activated, on the other hand, then the method 800 proceeds to step S803 where it is determined whether a positive (upward) vertical displacement of the medical pump 100 relative to the patient 200a,b has occurred, whether a negative (downward) vertical displacement of the medical pump 100 relative to the patient 200a,b has occurred, or whether no vertical displacement has occurred. The determination may be made, again for example, by the processor 190. If it determined that no vertical displacement has occurred, the method 800 proceeds to step S850, where no action is taken. If it is determined that a positive vertical displacement has occurred, the method 800 proceeds to step S842 where a prompt is displayed on a GUI 140 of the medical device 100 asking for an input as to whether a make-up volume should be infused. If an affirmative input is received at the GUI 140 (i.e. that a make-up volume should be infused), then the method 800 proceeds to step S852 where a make-up volume of fluid is delivered to compensate for the time of no flow based on the amount of vertical displacement determined from the sensors of the medical pump 100. If a negative input is received at the GUI 140, then the method 800 proceeds to step S850, where no action is taken. If it is determined that a negative vertical displacement has occurred, the method proceeds to step S844, where a prompt is displayed on a GUI 140 of the medical device 100 asking for an input as to whether a period of low flow should be caused. If an affirmative input is received at the GUI 140 (i.e. that a period of low flow should be caused), then the method 800 proceeds to step S854 where a time of reduced flow is caused to compensate for the volume of unintended bolus based on the amount of vertical displacement determined from the sensors of the medical pump 100. If a negative input is received at the GUI 140, then the method 800 proceeds to step S850, where no action is taken.

The method 800 may be run continuously or repeatedly by the controller and/or processor of the medical pump 100.

### CONCLUSION

While the invention has been illustrated and described in detail in the drawings and foregoing description, such illustration and description is to be considered as exemplary and not restrictive in character. For example, certain embodiments described hereinabove may be combinable with other described embodiments and/or arranged in other ways (e.g., process elements may be performed in other sequences). Accordingly, it should be understood that only the preferred embodiment and variants thereof have been shown and described and that all changes and modifications that come within the spirit of the invention are desired to be protected.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

What is claimed is:

1. A system for measuring the vertical displacement of a medical pump, the system comprising:
a medical pump comprising:
a processor; and
at least one sensor in operable communication with the processor,
wherein the at least one sensor is configured to detect a change in height of the medical pump.

2. The system of claim 1, wherein the medical pump further comprises a memory, and wherein the memory stores data which correlates changes in height of a medical pump to at least one of a time of no flow and a volume of an unintended bolus.

3. The system of claim 2, wherein the processor determines at least one of a time of no flow and a volume of an unintended bolus based on the detected change in height of the medical pump.

4. The system of claim 1, wherein the at least one sensor includes a laser disposed on a bottom of the medical pump.

5. The system of claim 4, wherein the medical pump uses at least laser triangulation method with the laser to determine the change in height of the medical pump.

6. The system of claim 4, wherein the at least one sensor further includes an accelerometer disposed within or on the medical pump and configured to detect movement of the medical pump, and wherein the medical pump is configured to turn on the laser when movement of the medical pump is detected using the accelerometer.

7. The system of claim 1, wherein the medical pump is a syringe pump and the at least one sensor includes a pressure sensor disposed in a drive head of the syringe pump which interfaces with a syringe plunger to determine an internal pressure of fluid in the patient line during therapy.

8. The system of claim 7, wherein a change in internal pressure of fluid in the patient line is used to further determine or confirm at least one of a time of no flow and a volume of an unintended bolus.

9. The system of claim 1, wherein the at least one sensor includes an accelerometer disposed in the medical pump and configured to determine the vertical displacement of the medical pump.

10. The system of claim 1, wherein the medical pump further comprises a graphical user interface configured to display at least one of a change in relative height of the medical pump, a time of no flow, a missing volume, and a volume of unintended bolus.

11. The system of claim 10, wherein graphical user interface further provides a user input to allow an operator to adjust an ongoing therapy based on the change in relative height of the medical pump.

12. The system of claim 10, wherein the medical pump is configured to cause an adjustment to an ongoing therapy to counteract effects of the change in relative height after a set period of time unless a user input is received.

13. The system of claim 1, wherein the medical pump is configured to include a transport mode.

14. The system of claim 13, wherein the transport mode causes the medical pump to automatically adjust infusion therapy to counteract effects of the change in relative height at a fastest processing speed of the processor based on data which correlates changes in height of a medical pump to at least one of a time of no flow and a volume of an unintended bolus.

15. The system of claim 13, wherein the medical pump further includes a graphical user interface configured to display when the transport mode is active.

16. The system of claim 13, wherein the transport mode is configured to run for a set period of time.

17. The system of claim 1, wherein the at least one sensor is configured to detect a change in relative height of a patient connected to the medical pump during therapy.

18. The system of claim 17, wherein the medical pump is configured to generate at least one of an alarm, an alert, a notification, or a pop-up when a change in the relative height of the patient is detected.

19. The system of claim 1, wherein the medical pump further comprises a memory which stores data specific to neonatal patients which correlates changes in height of a medical pump to at least one of a time of no flow and a volume of an unintended bolus.

20. The system of claim 19, wherein the medical pump is configurable to run in a neonatal patient mode, wherein the medical pump utilizes the data specific to neonatal patients while in the neonatal patient mode.
